(19) United States
(12) Patent Application Publication
(10) Pub. No.: US 2009/0240106 A1
(43) Pub. Date: Sep. 24, 2009

(54) Endoscope With a Stimulating Electrode For Peripheral Nerve Blocks Under Direct Vision

(75) Inventor: Daneshvari R. Solanki, League City, TX (US)

Correspondence Address: ROBERT W STROZIER, P.L.L.C, PO BOX 429, BELLAIRE, TX, 77402-0429, US

(73) Assignee: BOARD OF REGENTS, THE UNIVERSITY OF TEXAS SYSTEM, Austin, TX (US)

(21) Appl. No.: 12/398,312

(22) Filed: Mar. 5, 2009

Related U.S. Application Data
(60) Provisional application No. 61/034,092, filed on Mar. 5, 2008.

(51) Int. Cl. A61N 1/36; A61B 1/07

(52) U.S. Cl. 600/104; 607/46

(57) ABSTRACT

A nerve blocking endoscopic apparatus is disclosed having a stimulating electrode disposed at its distal end to aid in proper identification and placement of the apparatus adjacent a nerve to be blocked and to methods for making and using same.

### RELATED APPLICATIONS

The present invention claims the benefit and priority to U.S. Provisional Patent Application Ser. No. 61/034,092 filed 5 Mar. 2008 (Mar. 5, 2008).

### BACKGROUND OF THE INVENTION

1. Field of the Invention

Embodiments of the present invention relate to an endoscope for peripheral nerve blocks under direct observational control via the endoscope and to methods for making and using same.

More particularly, embodiments of the present invention relate to an endoscope including a stimulating electrode disposed at its distal end. Such an endoscope, when properly positioned under direct observational control via the endoscope, permits effective endoscope placement and electrode stimulation of the nerve for achieving effective peripheral nerve blocks under direct control using a local anesthetic agent. The present invention also relates to methods for making and using same.

2. Description of the Related Art

The technology of video assisted procedures using endoscopy is available and is not new. Video assisted endoscopic procedures are used for vein harvesting, thyroid surgery, carpel tunnel release, etc. But all these techniques require a surgical incision.

Presently the peripheral nerve blocks are done using indirect methods like paresthesias, peripheral nerve stimulation and ultrasound guided techniques.

However, there are no video assisted endoscopic procedures available at present for the performance of peripheral nerve blocks for regional anesthesia. Thus, there is a need in the art that is satisfied by this invention, an apparatus and method for performing video assisted endoscopic peripheral nerve blocks for regional anesthesia under direct vision or through the use of a video monitor.

### SUMMARY OF THE INVENTION

Embodiments of the present invention provide an endoscope including a stimulating electrode disposed at its distal end, where the endoscope, when properly positioned under direct observational control via the endoscope via the introducer, permits effective electrode placement adjacent to a nerve to be simulated for achieving effective peripheral nerve blocks. The endoscope may also include a fluid injection conduit having an opening at or near the distal end of the endoscope.

Embodiments of the present invention provide a method for performing a nerve block of a peripheral nerve including the steps of introducing an endoscope percutaneously without a surgical incision into an animal including a human. The endoscope includes an electrode disposed at or near its distal end and connected to a stimulation unit via a conducting conduit or wire. Once the endoscope has been inserted, the endoscope is pushed into a tissue of an animal including a human under direct visual control or visual control using a video monitor until the electrode is adjacent a peripheral nerve to be blocked. After the electrode is properly positioned, the nerve can be blocked using a local anesthetic injection through a fluid conduit to produce anesthesia so that a surgical procedure can then be performed. The conduit for administering anesthesia can also be used to place a stimulating catheter or a non-stimulating catheter to be left in place so that additional amounts of the anesthetic agents can be administered to the nerve to maintain the block or provide postoperative pain control. The endoscope can then be removed.

Embodiments of the present invention provide an endoscopic apparatus for locating peripheral nerve blocks including a body having a proximal end and a distal end. The apparatus also includes a light delivery assembly having one light delivery conduit or a plurality of light delivery conduits and one light receiving conduit or a plurality of light receiving conduits, where each light delivery conduit is connected to a light source and terminates in the distal end of the body, and where each light receiving conduit is connected to an image processing and terminates in the distal end of the body. The apparatus also includes a fluid delivery assembly including a fluid conduit terminating in an exit port near a distal end of the body and connected to fluid delivery unit adapted to deliver fluid through the fluid conduit. The apparatus also includes a nerve stimulating assembly including a nerve simulator unit having a first electrode disposed at or near the distal end of the body and connected via a first conducting conduit to the nerve stimulator unit, and a second electrode adapted to be placed on the skin of a patient and connected via a second conducting conduit to the nerve stimulator unit to produce a voltage between the first and second electrodes, where the voltage stimulates a nerve response when the first electrode is proximal a nerve. The distal end of the body is adapted to be inserted into an animal including a human, where the progress of the insertion is monitored by the image processing and display unit so that the distal end can be properly situated adjacent a nerve to be blocked and where the nerve simulator is adapted to properly identify the nerve to be blocked and where the fluid delivery assembly is adapted to deliver a nerve blocking agent to the identified nerve. In certain embodiments, the first electrode surrounds a portion of the body near its distal end. In other embodiments, the first electrode comprises a ring. In other embodiments, the ring is a solid ring, metal foil ring, or a wire mesh ring. In other embodiments the first electrode comprises at least one electrode element. In other embodiments, the first electrode comprises a plurality of electrode element. In other embodiments the first electrode comprises a plurality of electrode element equal spaced around an outside surface of the body near the distal end of the body. In other embodiments, the apparatus further includes a catheter adapted to be fed through the fluid conduit and left in place after removal of the endoscope. In other embodiments, the fluid delivery assembly includes a Y-connector so that the type of fluid introduced into the fluid conduit can be changed. In still other embodiments, the light delivery and receiving conduits comprises optical fibers or optical fiber bundles.

Embodiments of the present invention also provide a method for blocking a nerve including the step of inserting an endoscopic apparatus of this invention into a tissue site of an animal including a human. The method also includes the step of guiding the distal end of the apparatus by viewing progress of the insertion on the image processing and display unit. The method also includes the step of positioning the distal end of the endoscope adjacent to a peripheral nerve to be blocked. The method also includes the step stimulating the nerve with the stimulating electrode to identify the nerve and to ensure proper placement of the distal end of the apparatus. In certain embodiment, the method further includes the step administering a local anesthetic agent into the tissue site to block the identified nerve to produce anesthesia in the nerve so a surgical procedure can be subsequently performed. In other embodiments, the method further includes the step of inserting a catheter into the site through the fluid conduit and removing the apparatus so that the nerve can be blocked for a period of time after surgery.

### BRIEF DESCRIPTION OF THE DRAWINGS

The invention can be better understood with reference to the following detailed description together with the appended illustrative drawings in which like elements are numbered the same.

FIGS. 1A-B depict an embodiment of an endoscope of this invention including a ring type stimulating electrode disposed at or near a distal end of the endoscope.

FIGS. 1C-E depict another embodiment of an endoscope of this invention including a stimulating electrode comprising a set of electrode ends disposed at or near a distal end of the endoscope.

FIGS. 1F-G depict another embodiment of an endoscope of this invention including a mesh type stimulating electrode disposed at or near a distal end of the endoscope.

FIGS. 2A-C depict another embodiment of an endoscope of this invention including a retractable type stimulating electrode disposed at or near a distal end of the endoscope.

FIGS. 3A-F depict a method for producing an endoscope insertion site in an animal including a human: (A) depicts a catheter with the insertion needle; (B) depicts the removal of the needle and the insertion of a guide wire into and through the catheter; (C) depicts a dilator threaded down the guide wire to expand a tissue at the insertion site; (D) depicts a trocar including a balloon threaded down the guide wire onto the dilator to further expand the tissue; (E) depicts the trocar in place after removal of the dilator and guide wire and balloon inflation, and (F) depicts the removal of the guide wire and dilator and insertion of an endoscope of this invention.

FIGS. 4A-E depict method for producing an endoscope insertion site in an animal including a human: (A) depicts a catheter with the insertion needle; (B) depicts the removal of the needle and the insertion of a guide wire into and through the catheter; (C) depicts a dilator threaded down the guide wire to expand a tissue at the insertion site; (D) depicts removal of the guide wire and insertion of a trocar including a gas conduit for pressure tissue dissection; and (E) depicts the trocar in place and the replacement of the dilator and guide wire with an endoscope of this invention.

FIGS. 5A-E depict a method for inserting an endoscope into an insertion site of an animal: (A) depicts a catheter with the insertion needle; (B) depicts the removal of the needle and the insertion of a guide wire into and through the catheter; (C) depicts a dilator threaded down the guide wire to expand a tissue at the insertion site; (D) depicts a trocar including a distal balloon and gas tissue expansion conduit threaded down the guide wire replacing the dilator to further expand the tissue; and (E) depicts the replacement of a portion of the trocar with an endoscope of this invention.

FIG. 6 depict an embodiment of an endoscope of this invention including a distally disposed electrode placed proximal to a nerve to be blocked, where placement adjacent to the nerve is verified by electrode stimulation.

### DETAILED DESCRIPTION OF THE INVENTION

The inventor has found that a new endoscope apparatus can be constructed with a distally disposed electrode for performing safe, efficient and effective peripheral nerve blocking. The apparatus is designed to be inserted percutaneously into a tissue of an animal including a human as a conventional endoscope. The distal end is then positioned so that the electrode is located adjacent to a nerve to be blocked to perform a nerve block. The electrode is then activated to stimulate the nerve and ensure proper placement of endoscope adjacent the nerve so that anesthesia administration can be performed safely, efficiently and effectively for the surgical procedure. This process is performed without the need for a surgical incision unlike other endoscopic procedures.

The present invention broadly relates to an endoscope apparatus including a rigid or flexible body, a light source and a light conduit for transmitting light from the light source to a distal end of the body of the endoscope. The endoscope apparatus also includes a light receiving conduit having its proximal end disposed in or at the distal end of the endoscope body and its distal end in optical communication with a monitor for observing a location of the distal end of the endoscope body as it is positioned into a tissue of an animal including a human adjacent a peripheral nerve to be blocked, where proper endoscope positioning is confirmed by nerve stimulation via an electrode disposed at or near a distal end of the endoscope. The electrode is connected via a conducting conduit or wire and to an electrode stimulating unit, where the unit also includes a second conducting conduit or wire leading to a skin contact pad which acts as ground.

The present invention broadly relates to a method for blocking a nerve including the step of inserting an endoscope apparatus of this invention into a tissue of an animal including a human and positioning the distal end of the endoscope adjacent to a peripheral nerve to be blocked. The electrode disposed on or at the distal end is then turned on to stimulate of the nerve to ensure proper endoscope placement under direct visual or video assisted visual control. Once proper endoscope placement is confirmed, a local anesthetic agent is then injected into the tissue site to block the appropriate nerve to produce anesthesia in its distribution of the nerve so a surgical procedure can be subsequently performed. The local anesthetic administering like a catheter can be left in place so that the nerve can be blocked for a period of time after surgery. The endoscope can then be removed.

The present invention having the following general aspects that make it uniquely suited for performing nerve blocking: (1) the endoscope has a length between about 5″ and about 10″; (2) the endoscope can be rigid or flexible; (3) the endoscope can be a fiberoscope or fiberoscope fiber; (4) the endoscope is adapted to be easy to maneuver so that it can be positioned efficiently and effectively adjacent a location on a peripheral nerve; (5) the endoscope is adapted to have an excellent optical resolution; (6) the endoscope is adapted to be introduced percutaneously through a trocar or an introducer; (7) no surgical incision is needed to introduce the endoscope; (8) the trocar can optionally include a regulated side port for introduction of dilating medium for distension of the tissues if needed; (9) the trocar can optionally have a second port. This port can be used for introduction of the stimulating needle or placement of perineural catheter, placement of stimulating catheter. So, the nerve can be stimulated under direct vision or the catheter can be threaded under direct vision minimizing the chances of trauma to the nerves and the blood vessels; (10) the trocar is adapted to have capability for placing a deflated balloon and such a balloon when inflated can provide distension of the tissues so the nerves and the blood vessels can be easily visualized; (11) the endoscope also includes a anesthesia port for administering an anesthetic agent after proper placement of the endoscope; and (12) the endoscope includes a side port with a distal opening that can be used to inject the local anesthetic and placing a perineural catheter

The endoscope of this invention combines direct vision and a stimulating electrode like the stimulating needle to improve the efficacy of performing peripheral nerve blocks. In certain embodiment, the endoscope will have a bullet tip so it is atraumatic. The endoscope is adapted to permit visualization for proper electrode placement and stimulation under direct vision of the distally disposed electrode of the endoscope. With an appropriate response the nerve can be anesthetized by injection of a minimum amount of a local anesthetic under direct vision to produce the nerve block.

The endoscopes of this invention will also have other applications including, without limitation: (1) perineural catheter placement to reduce failed blocks and to permit pain control; (2) blood vessels will be visible, especially central veins. These can be cannulated under direct vision and help prevent complications like arterial punctures and pneumothorax etc. It can help in doing some interventional pain procedures under direct vision and help minimize exposure to X-ray radiation as these presently are done under fluoroscopic guidance (Endoscopes can also be used for surgery performed under the skin or muscles as well as the repair of the peripheral nerves); (3) the endoscopes can include distension of tissues using gas like carbon dioxide at low pressures of about 1 to about 8 mm Hg or other fluid. The tissue distension can also be accomplished using a balloon.

The peripheral nerve blocks at present are mostly done by placing percutaneous stimulating needles percutaneously based on the anatomic knowledge of the location of the nerves. Some institutions now use ultrasound guidance for performing peripheral nerve blocks. This requires the use of ultrasound gel and lack of it or improper contact with it makes it hard to visualize the nerves. So direct vision would be a great advantage and would greatly facilitate efficient and effective nerve blocking. Although endoscopes are available, none have been used for this technique. The endoscope having a stimulating electrode will allow us to locate the nerve, stimulate the nerve and anesthetize the nerve all under direct visual control. Such a method will also allow us to deal with variations in anatomy. Some times rescue blocks are necessary to have complete anesthesia. This endoscopic method will allow the anesthesiologist to perform such rescue blocks expeditiously. On an average at University of Texas Medical Branch in Galveston, TS, we perform between 50 and 100 peripheral nerve blocks for regional anesthesia per month. We perform between 80 and 100 pain blocks per month and more than 40-60 ventral vein cannulations per month.

Anesthesiologists do the peripheral blocks and place central venous lines. Interventional pain physicians perform interventional pain procedures under fluoroscopic guidance. Anesthesiologists, internists, surgeons and critical care physicians place the central venous catheters. Versatility of the endoscope and user friendly features will allow for accurate placement of the stimulating catheter or needle for vascular puncture.

Nerve blocks are performed for surgical anesthesia and postoperative analgesia. The endoscopic method with a stimulating electrode will allow one to localize the nerve under direct visual control and anaesthetize the chosen nerve. This can improve the accuracy of placement of the nerve block and decrease the chances of nerve damage. It will also decrease the time it takes to do the nerve block. The volume of local anesthetic required to produce anesthesia or analgesia of that nerve will be much less as it is placed directly on the target and this also decreases the chances of producing local anesthetic toxicity. In certain embodiments, the endoscope will have a bullet tip so it minimizes the chances of causing damage to the nerves and prevents the inadvertent puncture of a blood vessel, which could lead to systemic toxicity to other organs in the body.

Endoscopes of this Invention

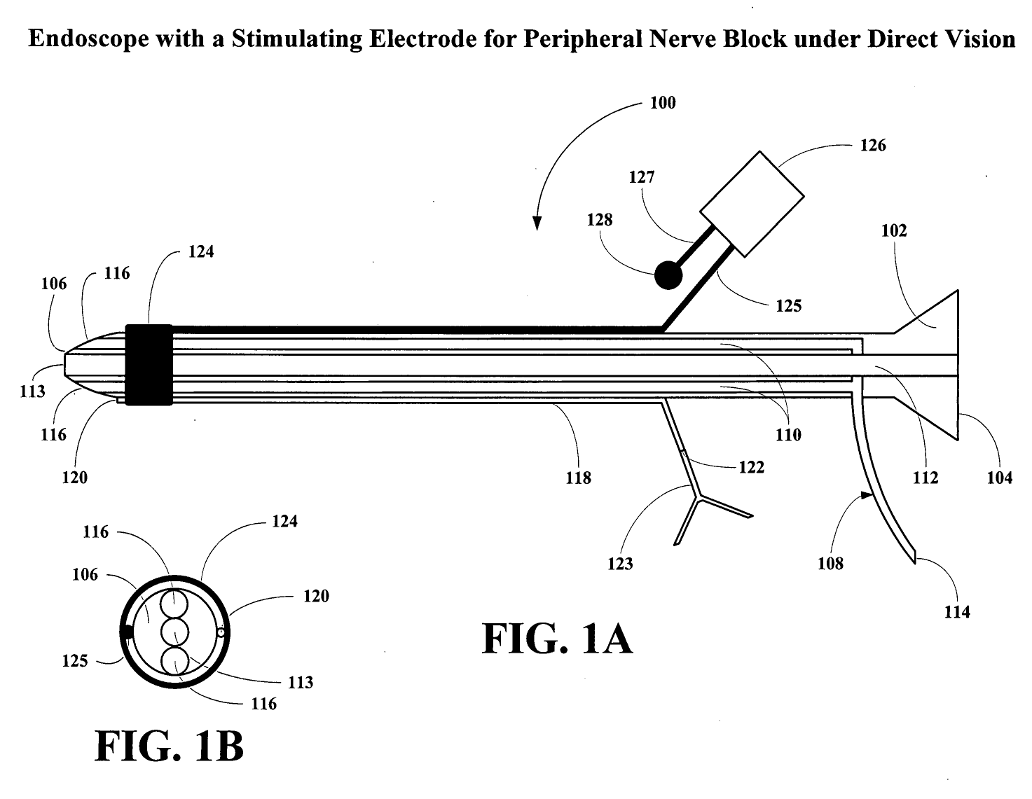

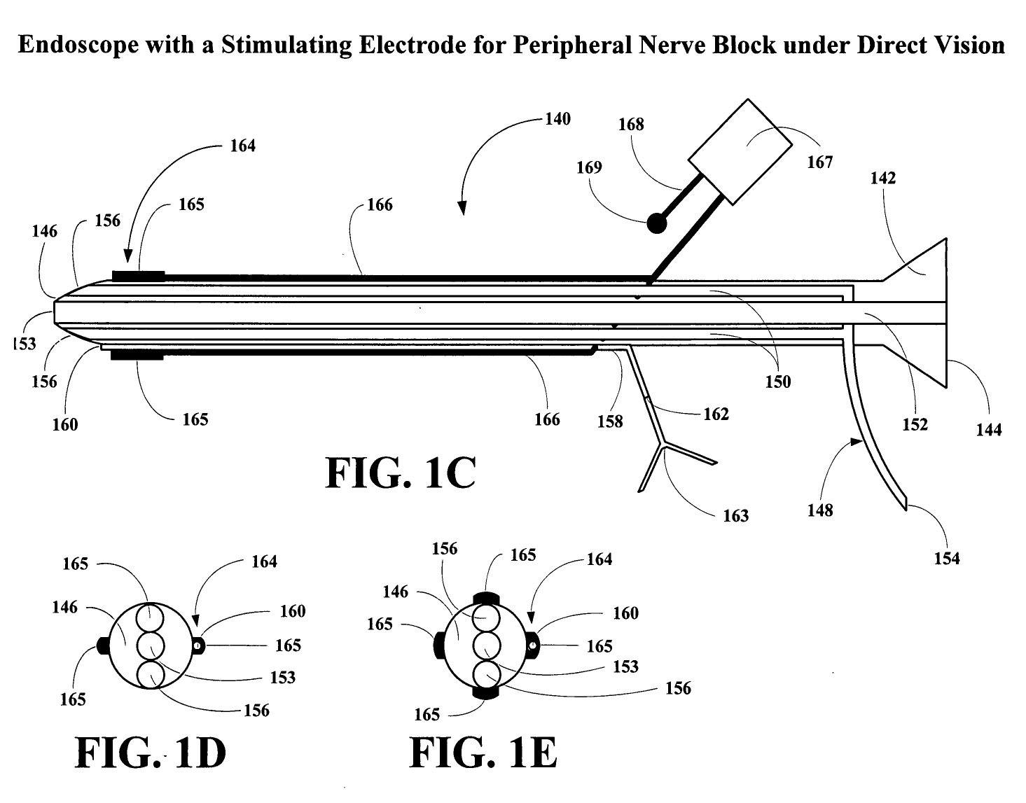

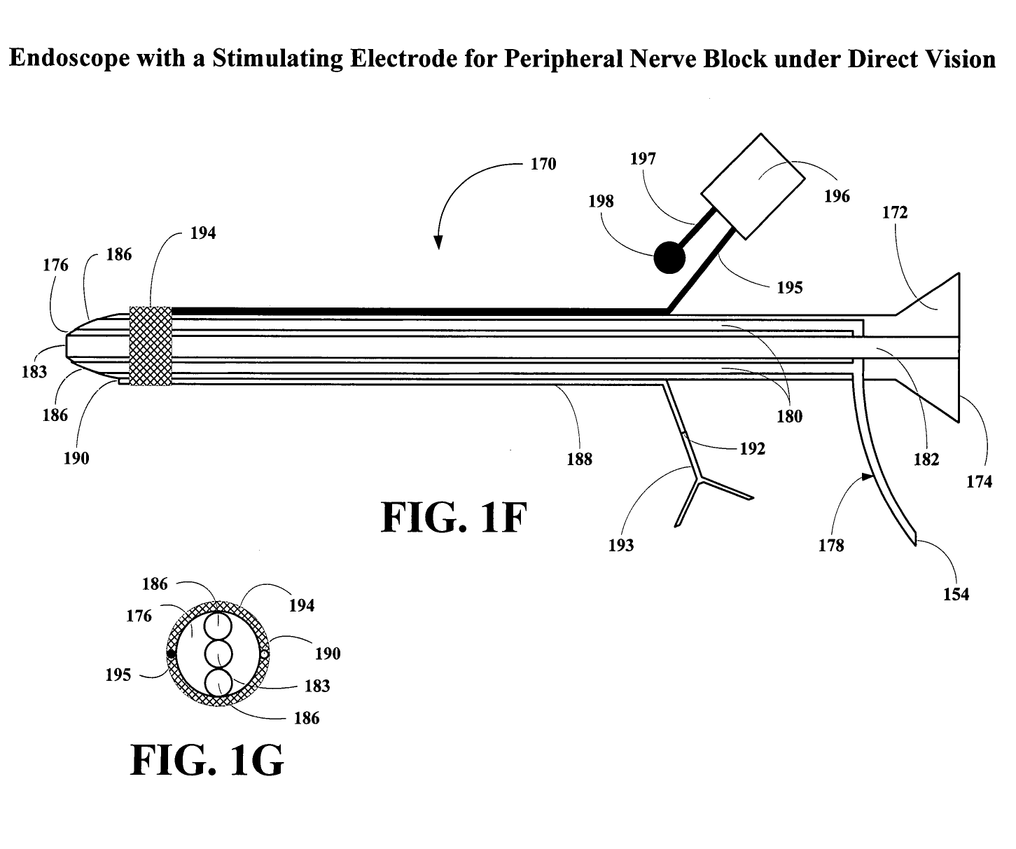

Referring now to FIGS. 1A&B, an embodiment of an apparatus of this invention, generally 100, is shown to include a body 102 having a proximal end 104 and a distal end 106. The apparatus 100 also includes a light delivery assembly 108 having two light delivery conduits 110 and a light receiving conduit 112, where the light delivery assembly 108 is connected to a light source (not shown) at its proximal end 114 and where the conduits 110 terminate in the distal end 106 of the body 102. Light exiting distal ends 116 of the conduits 110 are used to illuminate tissue as the endoscope 100 is inserted into a site of an animal including a human and a portion of the reflected light is received by a distal end 113 of the receiving conduit 112, where it can be directly viewed and/or travels to an image processing and display unit not shown. The apparatus 100 also includes a fluid conduit 118 terminating in an exit port 120 near a distal end 106 of the body 102. A proximal end 122 of the fluid conduit 118 is optionally fitted with a Y-connector 123 so that the type of fluid introduced into the fluid conduit 118 can be changed or a fluid, especially a gas, can be introduced to dilate the tissue so that the tissue can be visualized easily. The apparatus 100 also includes a ring shaped stimulating electrode 124 connected via a conducting conduit 125 to a nerve stimulator 126 having a second conducting conduit 127 including a second electrode 128 adapted to be placed on the skin to act as ground and to produce a voltage at the ring electrode 124. The electrode 124 can be composed of any conductor and can be of any size. In certain embodiments, the electrode 124 is a thin metal foil. Generally, the light delivery and receiving conduits comprises one or a plurality of optical fibers or optical fiber bundles. In certain embodiment, the light delivery and receiving conduits are a single fiber or microfiber.

For further details on the type of endoscopes that can be equipped with an electrode new block stimulator of this invention include at least the endoscopes disclosed in U.S. Pat. Nos. 7,150,752, 7,134,993, 6,793,622, 6,702,737, 6,699,183, 6,673,060, 6,641,528, 6,595,982, 6,522,933, 6,491,627, 6,482,148, 6,398,776, 6,236,876, 6,203,494, 6,030,360, 6,013,024, 5,960,145, 5,938,588, 5,916,147, 5,752,912, 5,681,263, 5,667,476, 5,575,755, 5,531,664, 5,512,035, 5,464,007, 5,448,989, 5,415,158, 5,396,880, 5,386,816, 5,381,782, 5,359,994, 5,347,989, 5,325,845, 5,301,656, 5,299,559, 5,176,126, 5,167,221, 5,005,558, 4,996,974, 4,967,732, 4,947,827, 4,941,454, 4,834,069, 4,796,607, 4,790,294, 4,787,369, 4,773,395, 4,762,119, 4,762,118, 4,750,477, 4,700,693, 4,688,555, 4,557,254, 4,499,895, 4,483,326, 4,432,349, 4,351,323, 4,294,233, and 4,203,430, incorporated herein by reference.

Referring now to FIGS. 1C-E, an embodiment of an apparatus of this invention, generally 140, is shown to include a body 142 having a proximal end 144 and a distal end 146. The apparatus 140 also includes a light delivery assembly 148 having two light delivery conduits 150 and a light receiving conduit 152, where the light delivery assembly 148 is connected to a light source (not shown) at its proximal end 154 and where the conduits 150 terminate in the distal end 146 of the body 142. Light exiting distal ends 156 of the conduits 150 is used to illuminate tissue as the endoscope is inserted into a site of an animal including a human and a portion of the reflected light is received by a distal end 153 the receiving conduit 152, where it travels to an image processing and display unit not shown. The apparatus 140 also includes a fluid conduit 158 terminating in a exit port 160 near a distal end 146 of the body 142. A proximal end 162 of the fluid conduit 158 is fitted with a Y-connector 163 so that the type of fluid introduced into the fluid conduit 158 can be changed or to use the fluid conduit to inject a component such as a gas to dilate the tissue. The apparatus 140 also includes a two element stimulating electrode 164 having two elements 165 connected via conducting conduits 166 to a nerve stimulator 167 having a second conducting conduit 168 including a second electrode 169 adapted to be placed on the skin to produce a voltage at the ring electrode 164 as shown in FIG. 1D. Looking at FIG. 1E, a second element stimulating electrode 164 is shown having four stimulating elements 165.

Referring now to FIGS. 1F&G, an embodiment of an apparatus of this invention, generally 170, is shown to include a body 172 having a proximal end 174 and a distal end 176. The apparatus 170 also includes a light delivery assembly 178 having two light delivery conduits 180 and a light receiving conduit 182, where the light delivery assembly 178 is connected to a light source (not shown) at its proximal end 184 and where the delivery conduits 180 terminate in the distal end 176 of the body 172. Light exiting distal ends 186 of the conduits 180 is used to illuminate tissue as the endoscope is inserted into a site of an animal including a human and a portion of the reflected light is received by a distal end 183 the receiving conduit 182, where it travels to an image processing and display unit not shown. The apparatus 170 also includes a fluid conduit 188 terminating in a exit port 190 near a distal end 176 of the body 172. A proximal end 192 of the fluid conduit 188 is fitted with a Y-connector 193 so that the type of fluid introduced into the fluid conduit 188 can be changed or to use the fluid conduit to withdraw fluids from the site. The apparatus 100 also includes a ring shaped mesh stimulating electrode 194 connected via a conducting conduit 195 to a nerve stimulator 196 having a second conducting conduit 197 including a second electrode 198 adapted to be placed on the skin to produce a voltage at the mesh electrode 194.

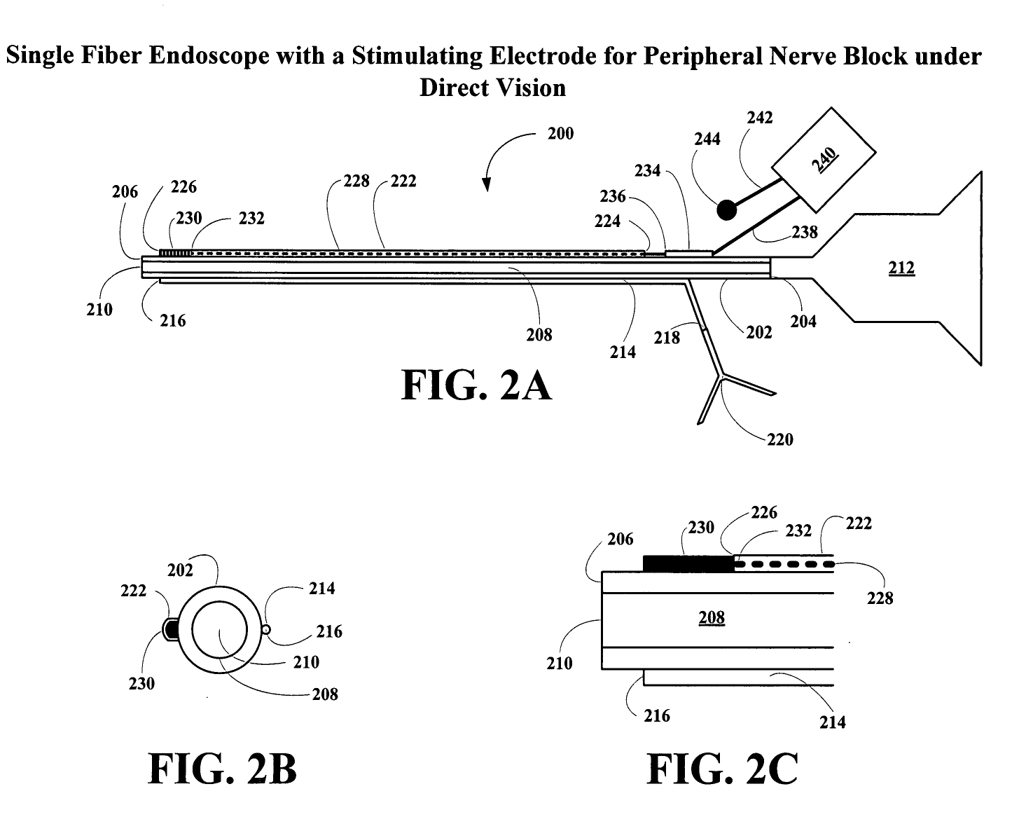

Referring now to FIGS. 2A-C, an embodiment of an apparatus of this invention, generally 200, is shown to include a body 202 having a proximal end 204 and a distal end 206. The apparatus 200 also includes a single optical fiber 208 adapted to deliver incident light to and receive reflected light from a tissue site into which the body 202 is inserted. Light exits through a distal end 210 of the fiber 208 and illuminates tissue as the endoscope 200 is inserted into a site of an animal including a human. A portion of incident light is reflected back into fiber 208 for subsequence analysis and conversion into an image with a light analyzing and image viewing unit 212. The apparatus 200 also includes a fluid conduit 214 terminating in an exit port 216 near the distal end 206 of the body 202. A proximal end 218 of the fluid conduit 214 is optionally fitted with a Y-connector 220 so that the type of fluid introduced into the fluid conduit 218 can be changed or a fluid, especially a gas, can be introduced to dilate the tissue so that the tissue can be visualized easily.

The apparatus 200 also includes an electrode housing 222 having a proximal end 224 and a distal end 226. Threaded through the housing 222 is a conducting member 228. The conducting member 228 includes an electrode 230 at its distal end 232 and is connected to an electrode extension and retraction unit 234 at its proximal end 236. The electrode extension and retraction unit 234 is adapted to shield the electrode 230 during endoscope 200 insertion and to extend the electrode 230 once the distal end 206 is positioned adjacent a nerve to be stimulated. The extension and retraction unit 234 is connected via a conducting conduit 238 to a nerve stimulator 240 having a second conducting conduit 242 including a second electrode 244 adapted to be placed on the skin to act as ground and to produce a volt difference across the electrodes 230 and 244.

Once the distal end 206 of the endoscope 200 is positioned relying on images or direct views through the fiber 208, the electrode 230 is extended and voltage is applied across the electrode 230 and 244 to stimulate the nerve. If the correct nerve is stimulated evidenced by flex action, then the electrode 230 can be retracted and anesthesia introduced to the site via the fluid conduit 214. The electrodes 230 and 244 and the conducting member can be composed of any conductor and can be of any size. The extension and retraction unit 234 can be a manual device or an electromechanical device that is adapted to push and pull the electrode 230 so that it can be extended or retracted.

Method for Using the Endoscopes of this Invention

Scenario No. 1

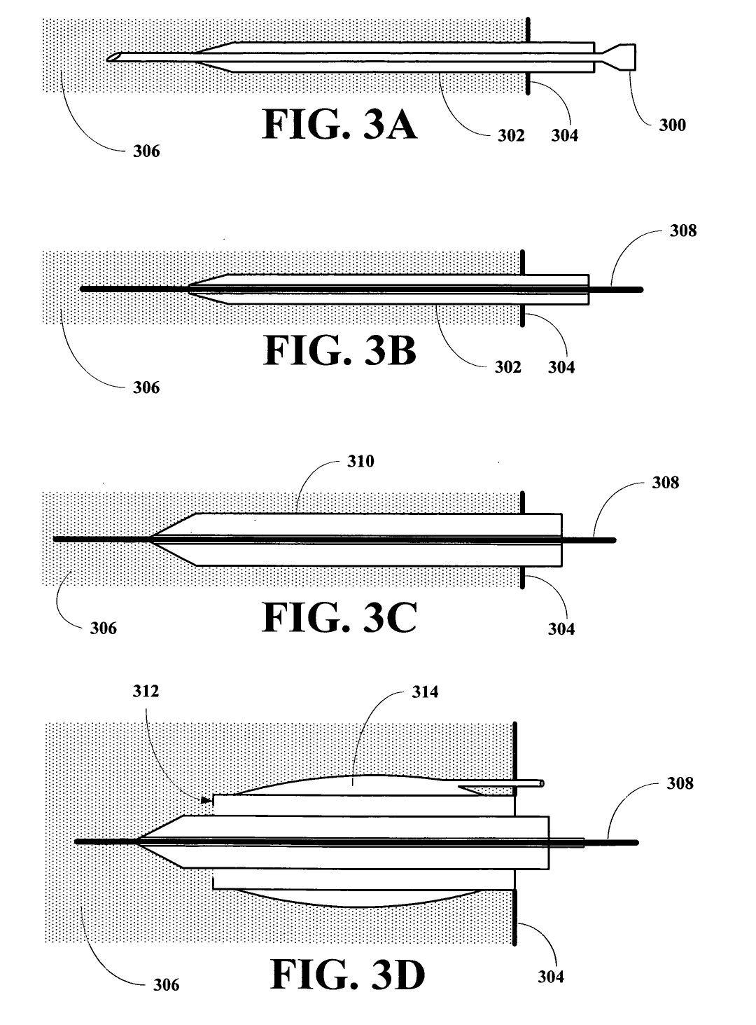

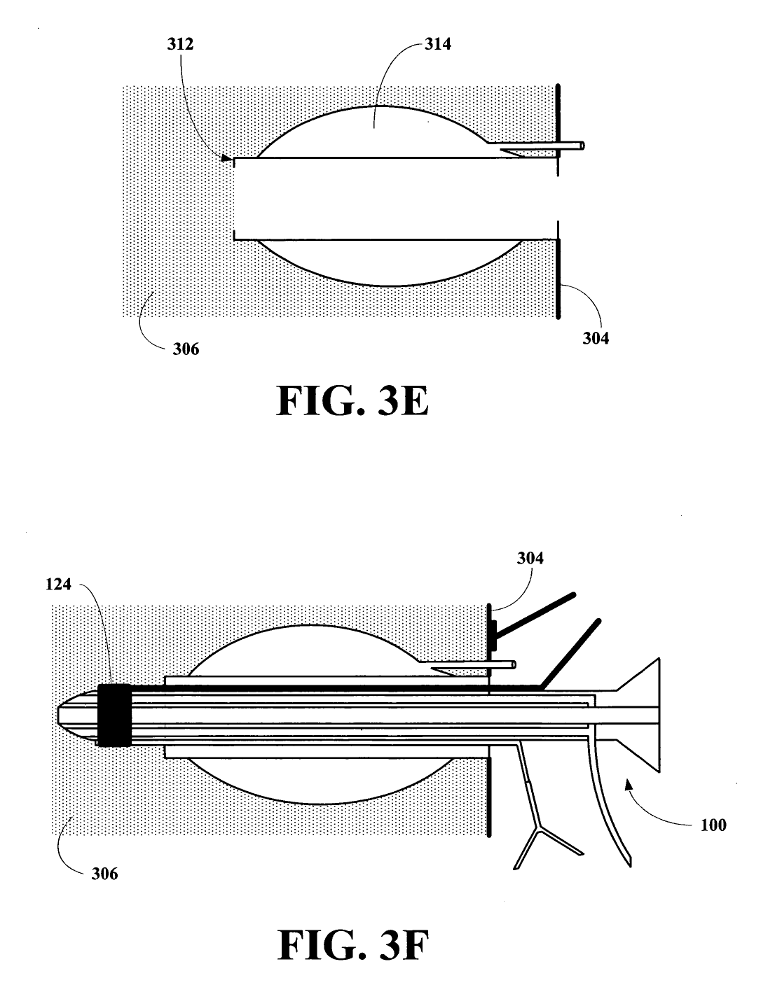

Referring now to FIGS. 3A-F, an embodiment of the method of this invention is shown. Looking at FIG. 3A, the method includes the step of inserting a needle 300 carrying a catheter 302 such as an angiocath through a skin 304 at a tissue site 306 of an animal including a human. Once properly placed, the needle 300 in removed and a guide wire 308 is threaded through the catheter 302 as shown in FIG. 3B. Next, the catheter 302 is removed and a dilator 310 is placed over the wire 308 and pushed into the site 306 to distend the tissue as shown in FIG. 3C. After distending the tissue using the dilator 310, a trocar 312 having a balloon 314 is placed over the dilator 310 and pushed into place to further distend the tissue as shown in FIG. 3D. The guide wire 308 and dilator 310 are then removed leaving the trocar 312 behind and the balloon 314 is inflated to further distend the tissue as shown in FIG. 3E. Next, an endoscope or a fiberoscope of this invention 100 including a ring electrode 124 is inserted through the trocar 312 to provide the ability of directly or through a monitor to visualize the structures and to permit proper positioning of the electrode on the endoscope as shown in FIG. 3F.

Scenario No. 2

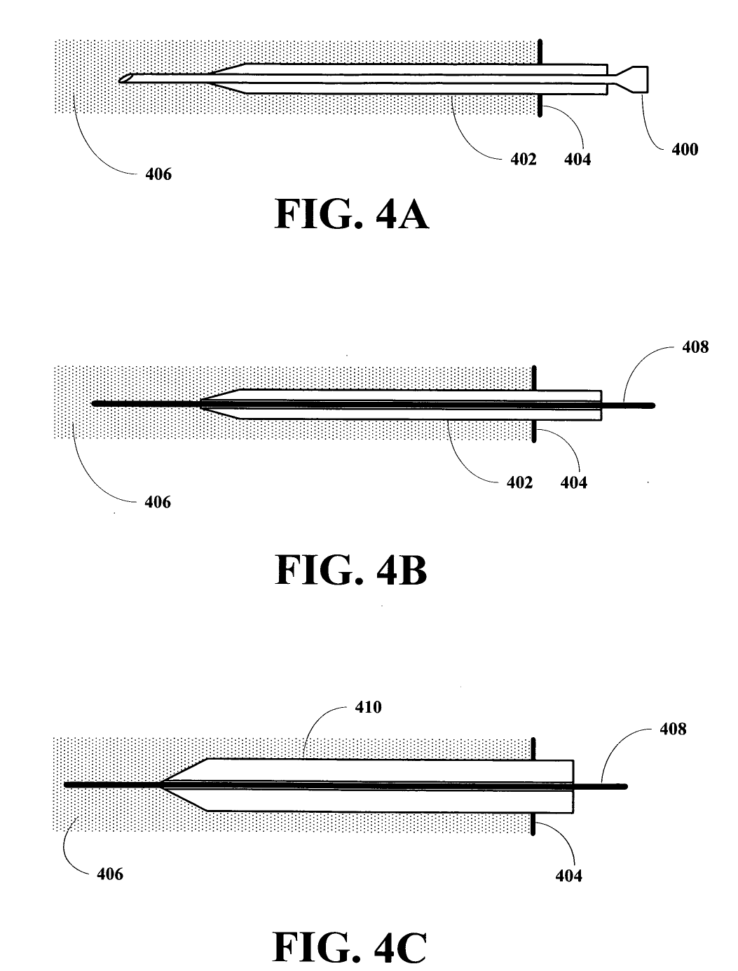

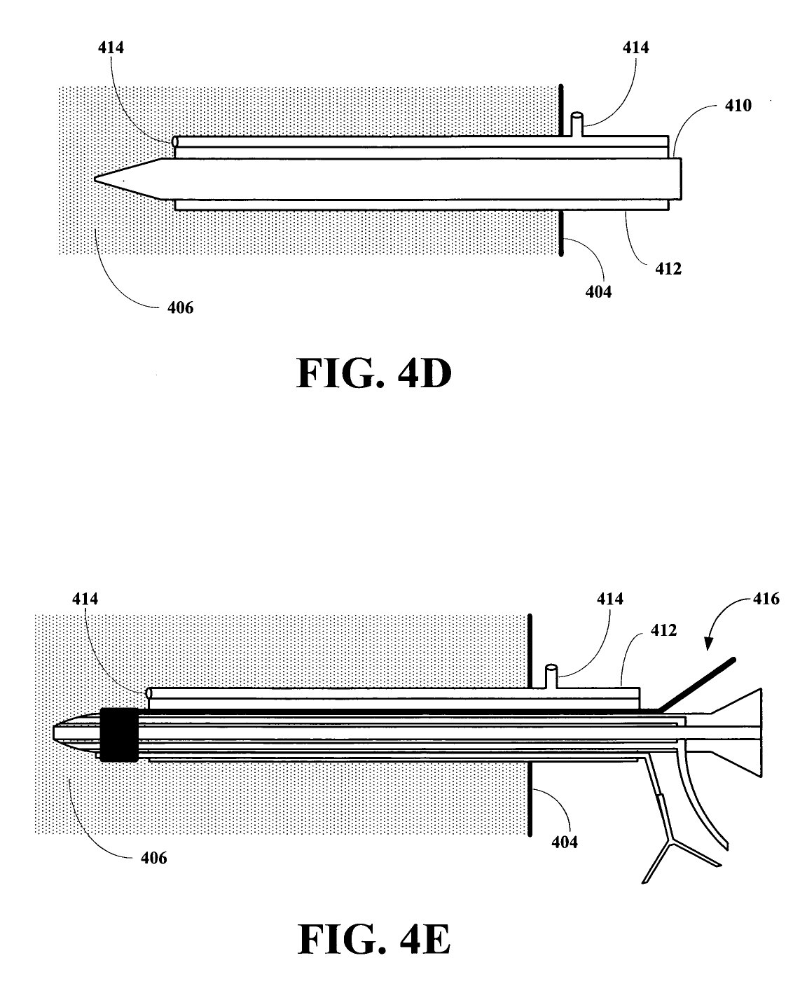

Referring now to FIGS. 4A-e, an embodiment of the method of this invention is shown. Looking at FIG. 4A, the method includes the step of inserting a needle 400 carrying a catheter 402 such as an angiocath through a skin at a site 404 of a tissue 406 of an animal including an human. Once properly placed, the needle 400 in removed and a guide wire 408 is threaded through the catheter 402 as shown in FIG. 4B. Next, the catheter 402 is removed and a dilator 410 is placed over the wire 408 and pushed into the tissue 406 to distend the tissue as shown in FIG. 4C. After distending the tissue using the dilator 410, a trocar 412 having a side port 414 is placed over the guide wire 408 and pushed into place as shown in FIG. 4D. Carbon dioxide is then insufflated at a pressure between about 4 and about 8 mm Hg through the side port 414 to distend the tissue. Next, the dilator 410 is removed and an endoscope or a fiberoscope of this invention 416 is inserted through the trocar 412 to visualize the structures and to permit proper positioning of the electrode on the endoscope as shown in FIG. 4E.

Scenario No. 3

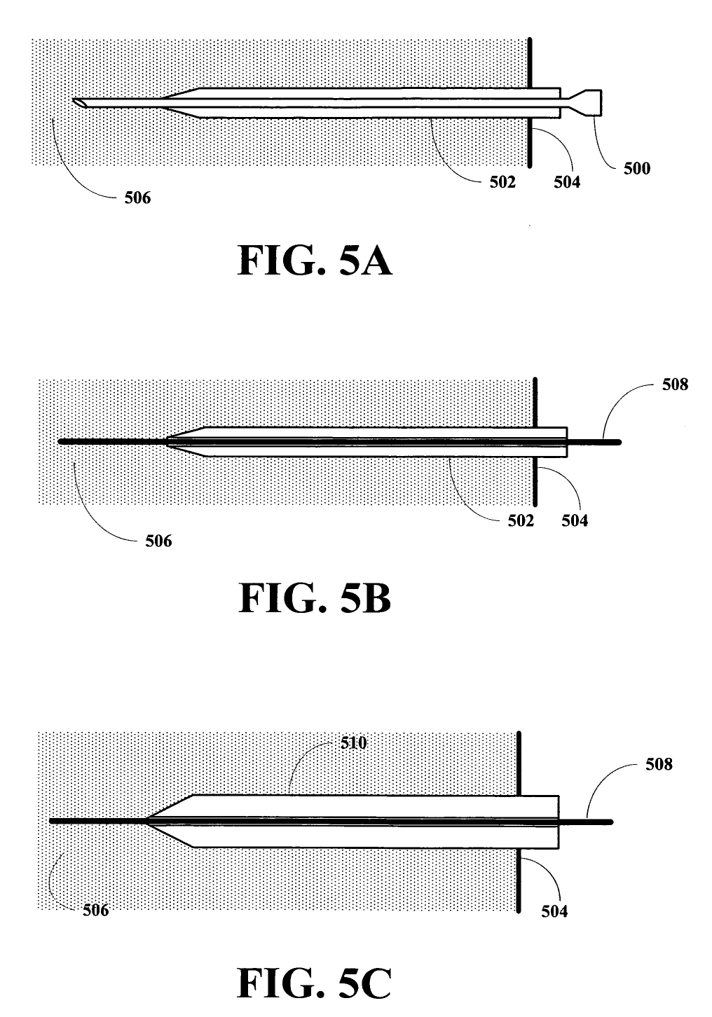

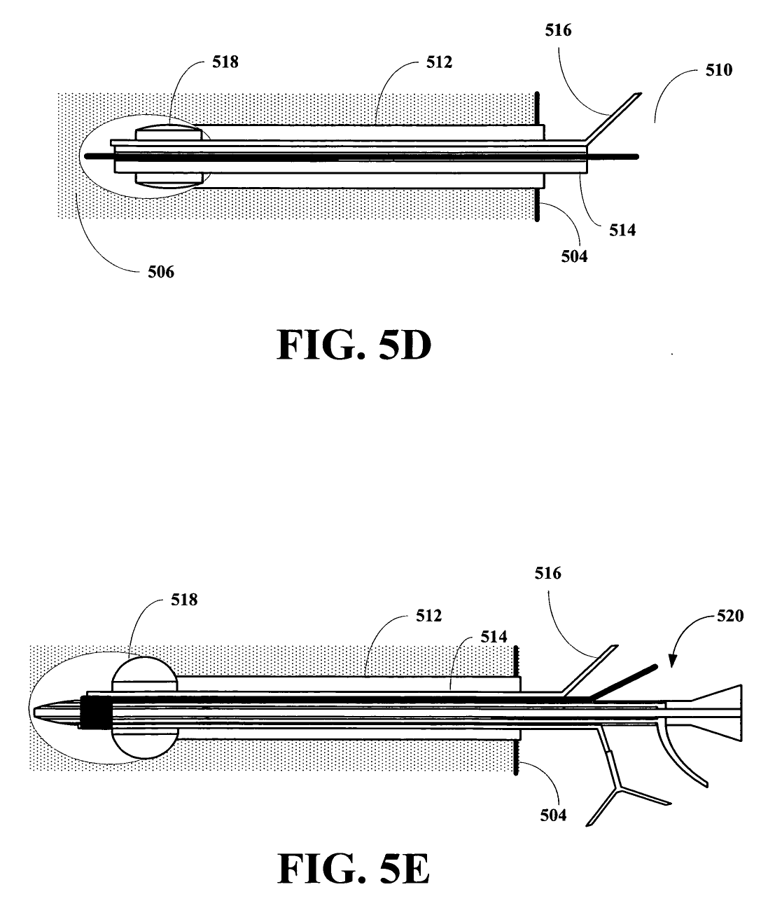

Referring now to FIGS. 5A-E, an embodiment of the method of this invention is shown. Looking at FIG. 5A, the method includes the step of inserting a needle 500 carrying a catheter 502 such as an angiocath through a skin at a tissue site 504 of a tissue 506 of an animal including an human. Once properly placed, the needle 500 in removed and a guide wire 508 is threaded through the catheter 502 as shown in FIG. 5B. Next, the catheter 502 is removed and a dilator 510 is placed over the wire 508 and pushed into the tissue 506 to distend the tissue as shown in FIG. 5C. After distending the tissue using the dilator 510, an introducer 512 is passed over the wire 508, and then a trocar 514 having a side port 516 and a circular balloon 518 is introduced over the introducer 512 into the tissue 506 and insufflate carbon dioxide through the side port 514 using a pressure between about 4 and about 8 mm Hg pressure to create an optical cavity. The balloon 518 is then inflated to further distend the tissue 506. Next, the wire 508 is then removed and a flexible endoscope of this invention 520 is passed through the trocar 514 and introducer 512 to visualize the structures and to permit proper positioning of the electrode on the endoscope as shown in FIG. 5E.

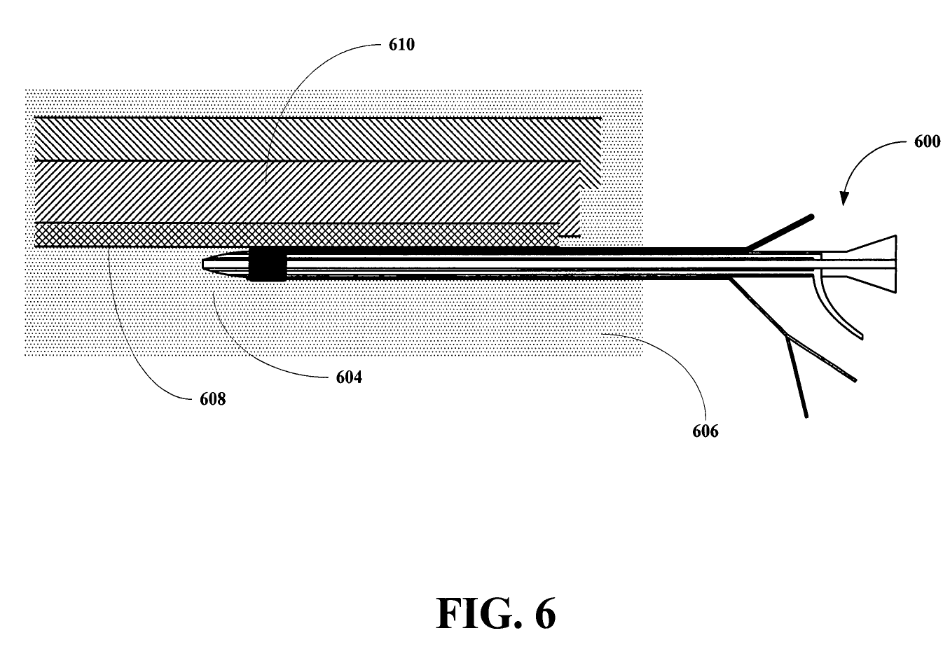

Referring now to FIG. 6, an embodiment of the apparatus, generally 600, is shown positioned adjacent a site 602 of a peripheral nerve 604. The apparatus 600 is located in a tissue 606 showing an artery 608 and a vein 610 traveling near the nerve 604.

All references cited herein are incorporated by reference. Although the invention has been disclosed with reference to its preferred embodiments, from reading this description those of skill in the art may appreciate changes and modification that may be made which do not depart from the scope and spirit of the invention as described above and claimed hereafter.

We claim:

1. An endoscopic apparatus for locating peripheral nerve blocks comprising:
a body having a proximal end and a distal end;
a light delivery assembly having one light delivery conduit or a plurality of light delivery conduits and one light receiving conduit or a plurality of light receiving conduits, where each light delivery conduit is connected to a light source and terminates in the distal end of the body, and where each light receiving conduit is connected to an image processing and terminates in the distal end of the body;
a fluid delivery assembly including a fluid conduit terminating in an exit port near a distal end of the body and connected to fluid delivery unit adapted to deliver fluid through the fluid conduit;
a nerve stimulating assembly comprising:
a nerve simulator unit including:
a first electrode disposed at or near the distal end of the body and connected via a first conducting conduit to the nerve stimulator unit, and
a second electrode adapted to be placed on the skin of a patient and connected via a second conducting conduit to the nerve stimulator unit to produce a voltage between the first and second electrodes, where the voltage stimulates a nerve response when the first electrode is proximal a nerve, and
where the distal end of the body is adapted to be inserted into an animal including a human, where the progress of the insertion is monitored by the image processing and display unit so that the distal end can be properly situated adjacent a nerve to be blocked and where the nerve simulator is adapted to properly identify the nerve to be blocked and where the fluid delivery assembly is adapted to deliver a nerve blocking agent to the identified nerve.

2. The apparatus of claim 1, wherein the first electrode surrounds a portion of the body near its distal end.

3. The apparatus of claim 1, wherein the first electrode comprises a ring.

4. The apparatus of claim 3, wherein the ring is a solid ring or a wire mesh ring.

5. The apparatus of claim 1, wherein the first electrode comprises at least one electrode element.

6. The apparatus of claim 1, wherein the first electrode comprises a plurality of electrode element.

7. The apparatus of claim 1, wherein the first electrode comprises a plurality of electrode element equal spaced around an outside surface of the body near the distal end of the body.

8. The apparatus of claim 1, further comprising a catheter adapted to be fed through the fluid conduit and left in place after removal of the endoscope.

9. The apparatus of claim 1, wherein the fluid delivery assembly includes a Y-connector so that the type of fluid introduced into the fluid conduit can be changed.

10. The apparatus of claim 1, wherein the light delivery and receiving conduits comprises optical fibers or optical fiber bundles.

11. A method for blocking a nerve comprising the steps of:
inserting an endoscopic apparatus into a tissue site of an animal including a human, where the apparatus comprises:
a body having a proximal end and a distal end;
a light delivery assembly having one light delivery conduit or a plurality of light delivery conduits and one light receiving conduit or a plurality of light receiving conduits, where each light delivery conduit is connected to a light source and terminates in the distal end of the body, and where each light receiving conduit is connected to an image processing and display unit and terminates in the distal end of the body;
a fluid delivery assembly including a fluid conduit terminating in an exit port near a distal end of the body and connected to fluid delivery unit adapted to deliver fluid through the fluid conduit;
a nerve stimulating assembly comprising:
a nerve simulator unit including:
a first electrode disposed at or near the distal end of the body and connected via a first conducting conduit to the nerve stimulator unit, and
a second electrode adapted to be placed on the skin of a patient and connected via a second conducting conduit to the nerve stimulator unit to produce a voltage between the first and second electrodes, where the voltage stimulates a nerve response when the first electrode is proximal a nerve,
guiding the distal end of the apparatus by viewing progress of the insertion on the display unit of the image processing and display unit,
positioning the distal end of the endoscope adjacent to a peripheral nerve to be blocked, and
stimulating the nerve with the stimulating electrode to identify the nerve and to ensure proper placement of the distal end of the apparatus.

12. The method of claim 11, further comprising the step:
administering a local anesthetic agent into the tissue site to block the identified nerve to produce anesthesia in the nerve so a surgical procedure can be subsequently performed.

13. The method of claim 11, further comprising the step:
inserting a catheter into the site through the fluid conduit and
removing the apparatus so that the nerve can be blocked for a period of time after surgery.

14. The method of claim 11, wherein the first electrode surrounds a portion of the body near its distal end.

15. The method of claim 11, wherein the first electrode comprises a solid ring, a foil ring, or a wire mesh ring

16. The method of claim 11, wherein the first electrode comprises an electrode element or a plurality of electrode element.

17. The method of claim 16, wherein the first electrode comprises a plurality of electrode element equal spaced around an outside surface of the body near the distal end of the body.

18. The method of claim 11, further comprising a catheter adapted to be fed through the fluid conduit and left in place after removal of the endoscope.

19. The method of claim 11, wherein the fluid delivery assembly includes a Y-connector so that the type of fluid introduced into the fluid conduit can be changed.

20. The method of claim 11, wherein the light delivery and receiving conduits comprises optical fibers or optical fiber bundles.
